(19) United States
(12) Patent Application Publication
(10) Pub. No.: US 2021/0401540 A1
(43) Pub. Date: Dec. 30, 2021

(54) INSTANTANEOUS DENTAL IMPLANT SURGERY GUIDING DEVICE AND METHOD THEREOF

(71) Applicant: Chao-Lin LIU, New Taipei (TW)

(72) Inventor: Chao-Lin LIU, New Taipei (TW)

(21) Appl. No.: 16/916,123

(22) Filed: Jun. 30, 2020

(51) Int. Cl. A61C 1/08; A61C 8/00; A61B 6/03; A61B 6/14; A61C 5/80

(52) U.S. Cl. CPC A61C 1/084; A61C 8/0089; A61B 2090/3966; A61B 6/14; A61C 5/80; A61B 6/032

(57) ABSTRACT

Disclosed are an instantaneous dental implant surgery guiding device and a method thereof, of which a main structure includes a base, a receiving space, a sphere, a through hole, and a adjustor. With the above structure, a dentist places the base at a tooth missing site in a patient oral portion, and, after applying glue for fixing, carries out computer-tomographic scanning to allow the dentist to diagnose and assess, and then, the adjustor may be operated to drive the sphere to move in the receiving space in order to change angle and position of the through hole of the sphere, thereby allowing the dentist to apply an implant driller to make a drill along a predetermined route defined by the through hole of the sphere, enabling subsequent operations of expanding the implant route in a bone with drillers of different sizes and positioning the implant.

### TECHNICAL FIELD OF THE INVENTION

The present invention relates to an instantaneous dental implant surgery guiding device and a method thereof, which feature safe and accurate positioning, instantaneousness and convenience, time saving, and cost reduction.

### DESCRIPTION OF THE PRIOR ART

Dental implantation is a dental restoration technique that uses an artificial root or fixture to support an artificial tooth for missing tooth restoration. The artificial root is commonly referred to as an implant. The implant is surgically placed in the alveolar bone of a patient where the tooth is missing. The implant may become a substitute of a natural tooth root to act as a foundation for mounting a crown. Once the alveolar bone of the patient and the implant become integrated with each other, an artificial crown may be attached to the implant. Mounting a false tooth with an artificial root provides the patient with an occlusal force close to that of a natural tooth and also achieves comfortableness and aesthetics that are much better than those of a traditional false tooth or denture. With oral hygiene being properly kept after dental implantation and constant dental inspection being practiced, an implant may maintain usable and lasting for quite a long period of time and is consequentially an important measure for restoration of missing teeth.

A known way of dental implant is carried out, in either flap or flapless manner, by applying a drilling bit to make drills into the bone, forming an implanting route, into which an implant is subsequently screwed. This may have certain risk and danger hiding in such a process, there being chance that severe damage may be caused on the patient, which often cause issues of medical malpractices. Later, dental implant that involves dental guides and computer tomography has been developed. Such a technique exhibits excellent safety. The dental guides include a diagnosis guide and a surgical guide. Conventionally, to apply such a technique, a tooth mode of the patient must be produced first and a wax material is used to sculpture or form a tooth that is intended to fill up the space in the tooth mold where a tooth is missing. Then, a new tooth mold is cast by using a specific material, such as alginate and gypsum, and a tooth mold that matches the shape of the patient and has no missing tooth is obtained. A thin plastic plate is disposed on the no-missing-tooth tooth mold and a process of heating and vacuum compression is applied to attach the plastic plate to a surface of the tooth mold. After proper trimming, the tooth-missing site is attached with a lead plate to serve as a positioning plate for tomography. In this way, the plastic plate may be applied to the teeth of the patient and the plastic plate having a configuration of the tooth mold may act almost like a transparent “holder”.

Then, the patient is guided to wear a diagnosis guide in the form of the holder and tomographic imaging is carried out, so that the oral configuration and a marked tooth-missing site will be shown in a computer. Further, the diagnosis guide may be drilled with holes in which an X-ray opaque material may be filled, and a positioning marking of a predetermined implantation route can be obtained after tomography.

Once the operation of tomographic imaging has been completed, some dental clinics may send the tomographic data, together with the tooth mold of the patient, to a dental technician workshop. In the contemporary digitalized dentistry, some dental clinics may adopt intraoral scanners to directly scan and identify the oral condition of the patient, which is sent, together with the tomographic files, to a digitalized technician workshop for direct fabrication of a surgical guide. The contemporary technical workshops often apply three-dimensional printing to fabricate, through printing, the surgical guide. The dental clinic, upon receiving the surgical guide, carries out the tooth implantation surgery on the patient by positioning the implant against the surgical guide for subsequently placing the implant in the tooth-missing site in the patient's oral cavity at a correct angle and a correct position.

Such a conventional way of tooth implant suffers the following problems and drawbacks that require further improvement:

(1) It takes high cost and time to retain a technician workshop to carry out fabrication of a traditional or digitalized diagnosis or surgical guide.

(2) Pre-surgery preparations for fabrication of a holder like diagnosis guide, including tomographic assessment, wax sculpturing, repeated molding, and vacuum compression, are complicated and tedious processes, and consequentially, a patient has to wait for certain time before surgery can be carried out.

(3) For sake of convenience, some dentists do not use such diagnosis and surgical guides, and this increases the surgery risk for the patient.

(4) Dentistry digitalization is a worldwide trend, yet digital dentistry equipment is expensive in cost and complicated in operation, and requires operators be highly trained for operating computers, equipment, and professional software. Considering the necessary labor, facility, and expenditure, it is not possible for immediate realization of digitalization for all the dental clinics.

### SUMMARY OF THE INVENTION

The primary objective of the present invention is that an adjustor is operable to control a sphere arranged in a receiving space, so as to have a through hole of the sphere moved to correct angle and position and to measure a spatial height from a top end of the through hole to a gum for calculating a driller length necessary for drilling to a predetermined position inside a bone. After the adjustor is removed, a dentist applies an implant driller to make a drill along the through hole, so as to generate a precise implant route for sub sequentially placing an implant therein. The above structure is provided for combining a diagnosis guide and a surgical guide with each other to achieve effects of instantaneousness and convenience, time saving and cost reducing, and ensuring accuracy and safety of surgery.

A main structure of the present invention comprises: a base, the base comprising a receiving space, the receiving space being provided with a sphere therein, the sphere comprising a through hole formed therein, the through hole receiving therein an adjustor partly penetrating therethrough, the base being provided on two sides thereof with a plurality of sphere positioners, the sphere positioners penetrating from outside of the base into the receiving space to abut the sphere, the base being further provided with at least one base positioner. The above-discussed adjustor comprises an X-ray opaque material, while the base, the sphere, the sphere positioners, and the base positioner are all X-ray transmittable.

To carry out dental implanting on a tooth-missing patient, a dentist first places the base at an implantation site in the patient oral portion with the base positioner of the base positioned against neighboring teeth, and glue is applied to initially retain the base positioner, and then, a molding material is coated on and covering the base positioner and the neighboring teeth, and waiting until curing of the molding material to carry out computer-tomographic scanning on the patient oral area for determining whether to carry out adjusting of the through hole of the sphere in respect of position and angle thereof.

When it is determined, according to the result of computer-tomographic scanning, that adjusting is necessary, the sphere positioners that abut against the sphere are controlled such that the sphere positioners are caused to move away from the sphere and the sphere is no longer constrained thereby. As such, the dentist may then operate and control the adjustor that is partly penetrating and arranged in the through hole for adjusting the sphere in respect of position and angle in order to change the position and angle of the through hole formed in the sphere. After the adjusting is completed, the sphere adjustor is moved in a way opposite to what described above to re-abut against the sphere so as to constrain the sphere, and at this moment, the sphere is secured at a correct position obtained with such adjusting, and a spatial height from a top end of the through hole to a gum can be measured with scales provided on the adjustor, and a driller length necessary for drilling to a predetermined position in a bone can then be calculated. Finally, the adjustor is removed out of the through hole, and the dentist may then apply an implant driller to drill, in a direction of the through hole, into the alveolar bone at the tooth missing site in the patient oral area to obtain accurate and precise direction and depth of implanting. As such, costs of a series of operations for fabricating a tooth mold, a diagnosis guide, and a surgical guide can be saved, and a great amount of time spent in pre-operation processes of dental implant surgery is also saved, and there is no need to purchase and install digitalized equipment that is currently expensive and training of operators, to thereby achieve effects of safe and accurate positioning, instantaneousness and convenience, time saving, and cost reduction.

### BRIEF DESCRIPTION OF THE DRAWINGS

FIG. 1 is a perspective view showing a preferred embodiment.

FIG. 2 is an exploded view of the preferred embodiment.

FIG. 3 is a flow chart showing steps of operation of the preferred embodiment.

FIG. 4 is a schematic view illustrating coating of the preferred embodiment.

FIG. 5 is a schematic view illustrating dismounting of the preferred embodiment.

FIG. 6 is another schematic view illustrating dismounting of the preferred embodiment.

FIG. 7 is a schematic view illustrating angle adjusting of the preferred embodiment.

FIG. 8 is a schematic view illustrating frontward-rearward adjusting of the preferred embodiment.

FIG. 9 is another schematic view illustrating frontward-rearward adjusting of the preferred embodiment.

FIG. 10 is a schematic view illustrating leftward-rightward adjusting of the preferred embodiment.

FIG. 11 is a schematic view illustrating implanting of the preferred embodiment.

FIG. 12 is a schematic view illustrating fastening of another preferred embodiment.

FIG. 13 is a schematic view illustrating fastening of a further preferred embodiment.

FIG. 14 is a schematic view illustrating mounting of yet a further preferred embodiment.

### DETAILED DESCRIPTION OF THE PREFERRED EMBODIMENT

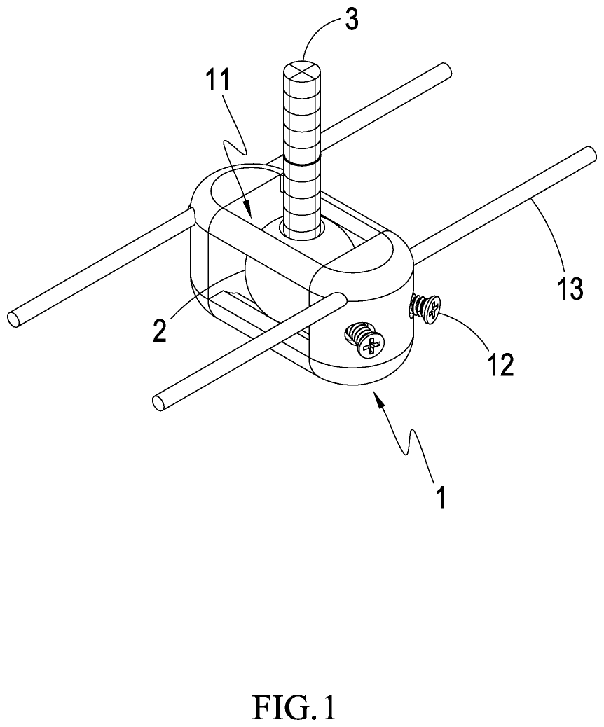

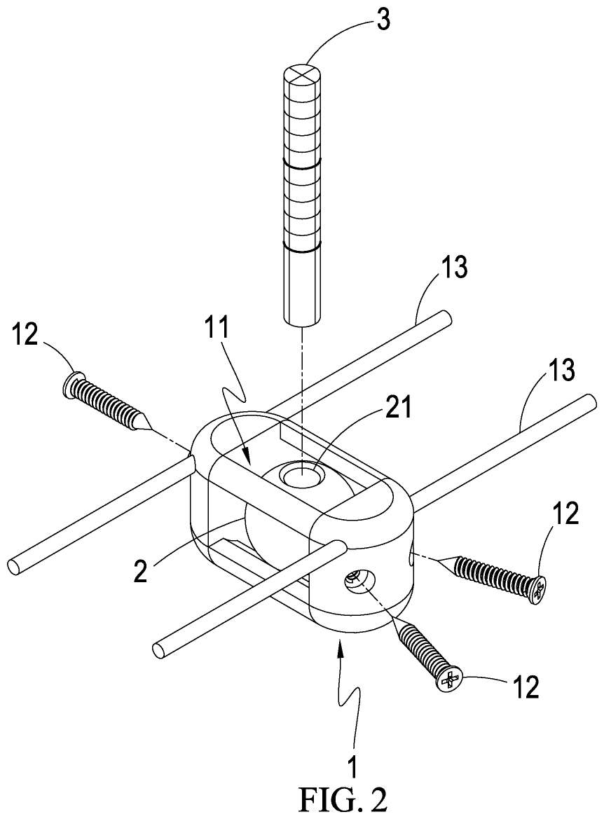

Referring to FIGS. 1 and 2, the present invention comprises:

a base 1, the base 1 comprising a receiving space 11 therein;

a sphere 2, the sphere 2 being arranged in the receiving space 11, the sphere 2 comprising a through hole 21 formed therein;

an adjustor 3, the adjustor 3 being partly penetrating and arranged in the through hole 21;

a plurality of sphere positioners 12, each of the sphere positioners 12 being arranged at one of two sides of the base 1 and penetrating from an outside of the base 1 into the receiving space 11 and abutting the sphere 2; and

at least one base positioner 13, the base positioner 13 being arranged on the base 1.

The sphere positioners 12 are exemplified as bolts of which the number is three. The base 1 is exemplified as a curved configuration. The adjustor 3 is exemplified as a bar including scales thereon. The adjustor 3 is made of an X-ray opaque material, while the base 1, the sphere 2, the sphere positioners 12, and the base positioner 13 are all X-ray transmittable. The base positioner 13 is exemplified as a bar that is slidable in the base 1.

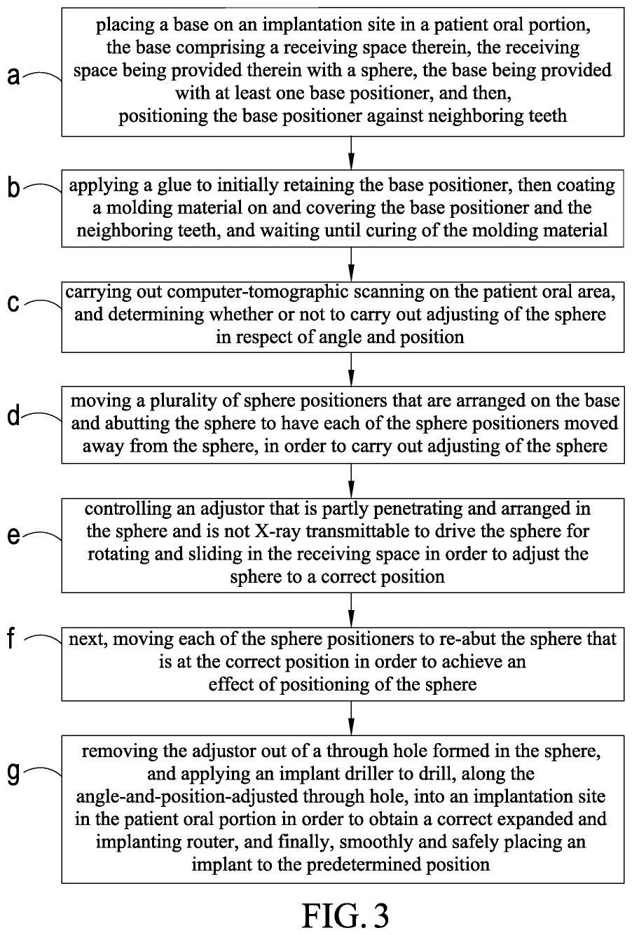

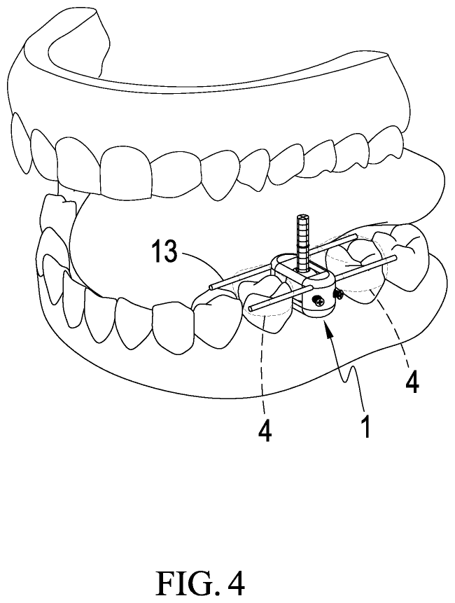

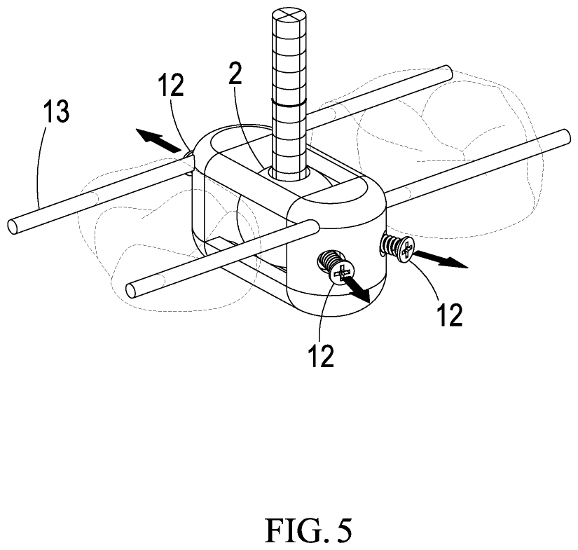

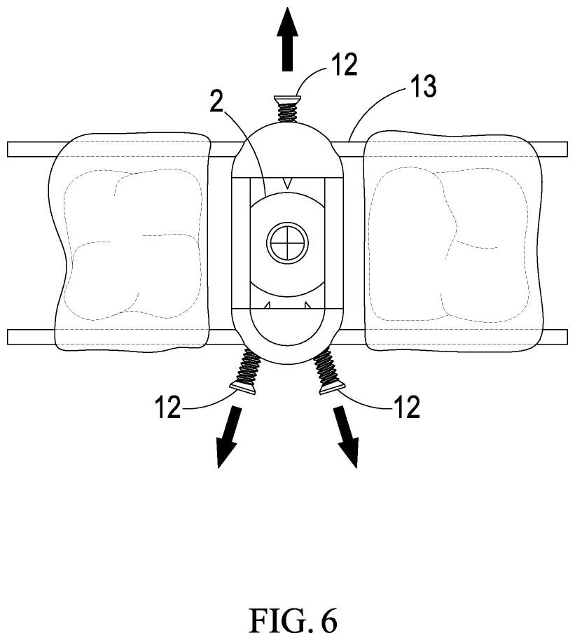

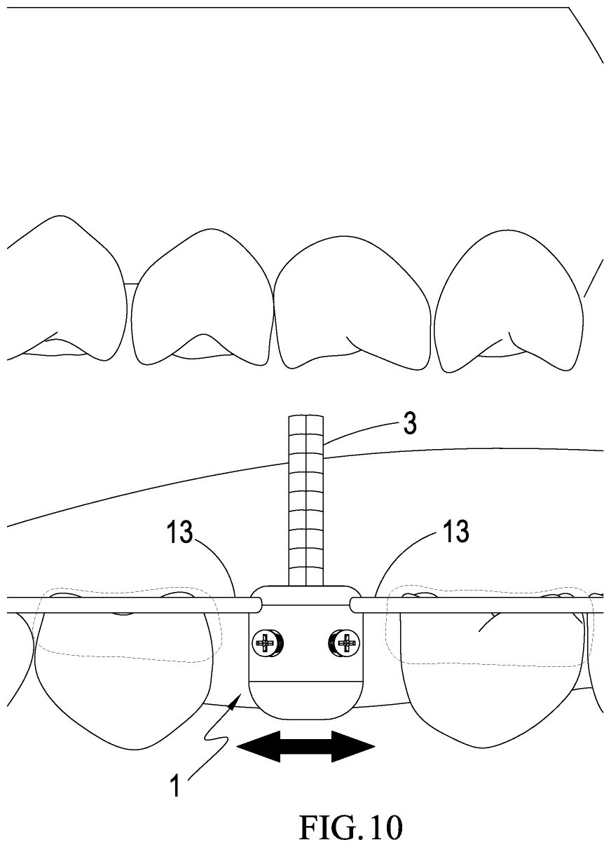

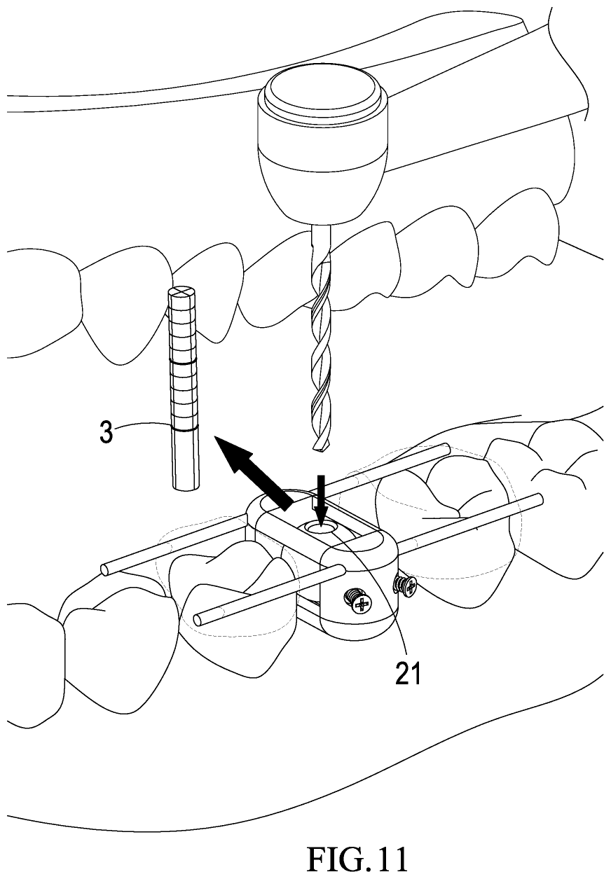

Referring to FIGS. 1-11, a method of operating or using the present invention comprises steps that includes:

(a) placing a base on an implantation site in a patient oral portion, the base comprising a receiving space therein, the receiving space being provided therein with a sphere, the base being provided with at least one base positioner, and then, positioning the base positioner against neighboring teeth;

(b) applying a glue to initially retaining the base positioner, then coating a molding material on and covering the base positioner and the neighboring teeth, and waiting until curing of the molding material;

(c) carrying out computer-tomographic scanning on the patient oral area, and determining whether or not to carry out adjusting of the sphere in respect of angle and position;

(d) moving a plurality of sphere positioners that are arranged on the base and abutting the sphere to have each of the sphere positioners moved away from the sphere, in order to carry out adjusting of the sphere;

(e) controlling an adjustor that is partly penetrating and arranged in the sphere and is not X-ray transmittable to drive the sphere for rotating and sliding in the receiving space in order to adjust the sphere to a correct position;

(f) moving each of the sphere positioners to re-abut the sphere that is at the correct position in order to achieve an effect of positioning of the sphere; and

(g) removing the adjustor out of a through hole formed in the sphere, and applying an implant driller to drill, along the angle-and-position-adjusted through hole, into an implantation site in the patient oral portion in order to obtain a correct expanded and implanting router, and finally, smoothly and safely placing an implant to the predetermined position.

When a tooth-missed patient is about to carry out a dental implant operation, the dentist first places the base 1 at a tooth-missing and to-be-implanted site in the patient oral portion, and at this time, glue is coated on the base positioner 13, wherein the glue is exemplified as a photo-curable resin and the photo-curable resin is a composite resin that is curable with irradiation of blue light of a predetermined wavelength, so as to initially and adhesively bond the base positioner 13 to left-side and right-side neighboring teeth. The base positioner 13 that is arranged on the base 1 is positioned against the teeth on the left side and the right side of the tooth-missing site, and then, a molding material 4 is coated on the base positioner 13 and the neighboring teeth, wherein the molding material 4 is exemplified as putty, but is not limited thereto. Once cured, the molding material 4 forms, in an inner side thereof, a raised-recessed configuration that matches the shape of the teeth of the patient, so as to get securely attached to the left-side and right-side neighboring teeth, while still maintaining certain elasticity after being cured and could be repeatedly attached to or removed from the left-side and right-side neighboring teeth.

Next, the dentist carries out computer-tomographic scanning on the oral portion of the patient, and makes assessment, according to analysis of scanning result, whether adjusting of the through hole 21 in respect of position and angle is necessary, such that if it is necessary to carry out the adjusting, the dentist removing the base 1 that has the molding material 4 coated thereon from the patient oral portion, and the base positioner 13 that is arranged on the base 1 and is temporarily bonded by the photo-curable resin to the neighboring teeth is consequentially also removed, and then, adjusting of angle and position of the through hole 21 of the sphere 2 can be carried out.

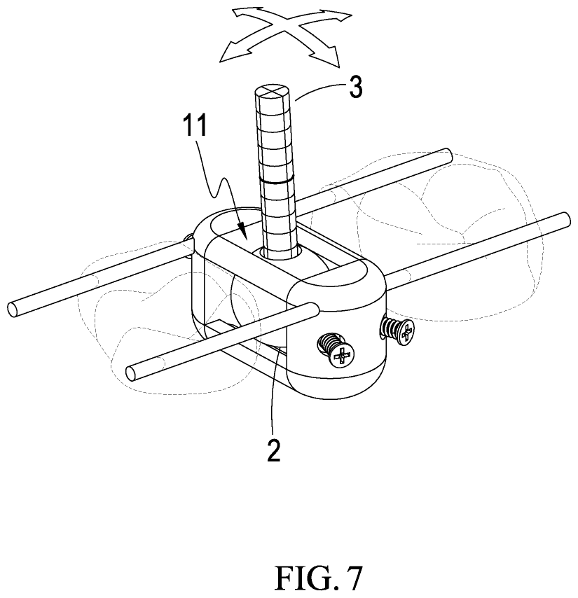

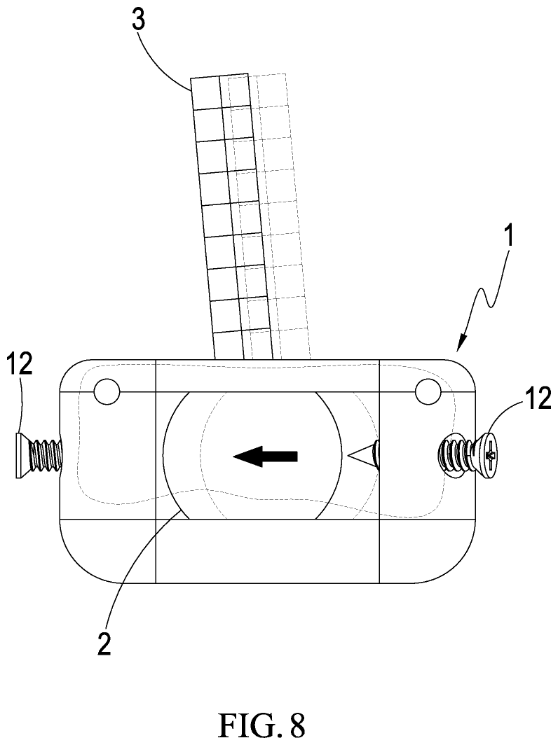

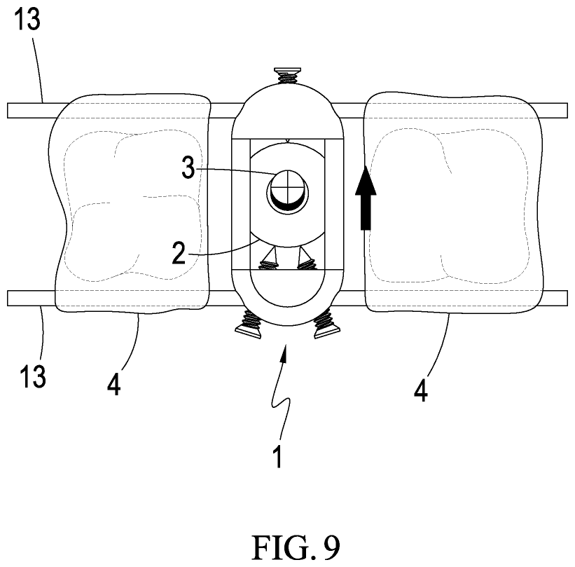

To do the adjusting, the sphere positioners 12 that abut the sphere 2 must be first dismounting from the sphere 2, making the sphere 2 no longer constrained by the sphere positioners 12, and then the dentist may control the adjustor 3 that is partly penetrating through and arranged in the through hole 21 to carry out adjusting of position and angle of the sphere 2, wherein by means of a structure of the adjustor 3, the sphere 2 is caused to move in the receiving space 11, such as that shown in FIGS. 8 and 9, in which an example where the sphere 2 is moved inwardly in a parallel manner by 1 mm is demonstrated, and the adjustor 3 is further operable to cause the sphere 2 to rotate in the receiving space 11 as shown in FIG. 7, so as to change the angular position of the sphere 2 and thus, making the through hole 21 formed in the sphere 2 change angular position.

Once the sphere 2 has been adjusted to position, the sphere positioners 12 can be moved against to positions of abutting the sphere 2, so as to secure the sphere 2 at correct angle and position, and then, in view of the material property of the molding material 4, the base 1 can be placed, as initially positioned, back to the to the tooth missing site in the patient oral portion, and since the molding material 4 has been already cured in a shape of matching the teeth of the patient, a complete effect of positioning can be immediately achieved for the base 1 upon the base 1 being placed back, and, together with the sphere 2 of which adjusting has been done, the through hole 21 formed in the sphere 2 is now set at a correct implantation angle and implantation position.

Finally, a spatial height from a top end of the through hole 21 to gum can be measured by using the scales provided on the adjustor 3, a driller length necessary for drilling to a predetermined position inside a bone can be calculated. The dentist only needs to withdraw the adjustor 3 out of the through hole 21 of the sphere 2 and then operates an implant driller 5 to drill, along the through hole 21, into the alveolar bone of the patient to complete positioning of route for first stage implantation of implant surgery. As such, there is no need to spend an extended period of time to repeatedly construct tooth molds, nor does it need to retain a technician workshop for making diagnosis and surgical guides that take a high cost, and, consequentially, advantageous effects of safe and accurate positioning, instantaneousness and convenience, time saving, and cost reduction can be achieved.

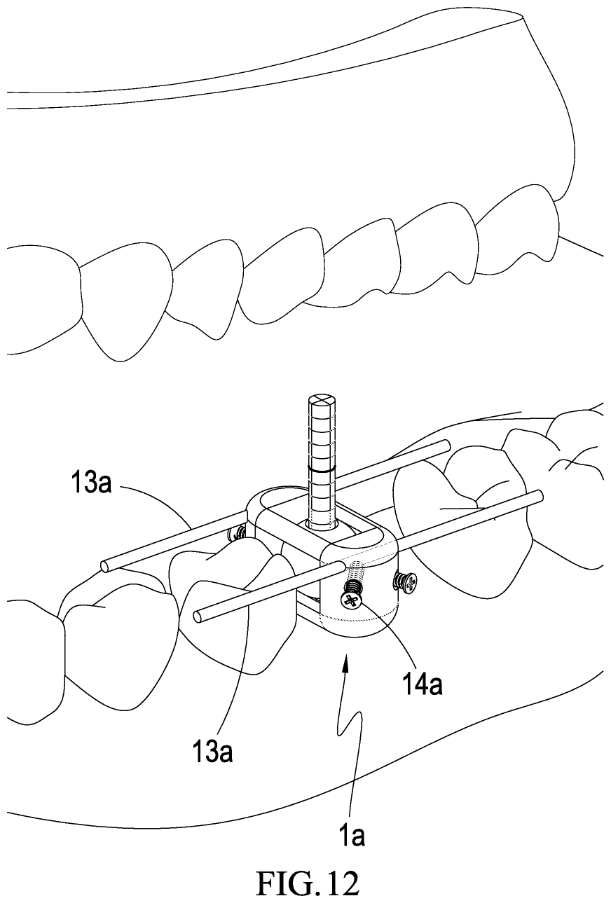

Referring to FIG. 12, the base 1a is provided with a fastening element 14a, and the fastening element 14a penetrates from an outside of the base 1a into an inside of the base 1a to abut the base positioner 13a, the fastening element 14a being exemplified as a bolt.

In the instant embodiment, Step (a1) can be carried out after Step (a) and is discussed as follows:

(a) placing a base on an implantation site in a patient oral portion, the base comprising a receiving space therein, the receiving space being provided therein with a sphere, the base being provided with at least one base positioner, and then, positioning the base positioner against neighboring teeth;

(a1) additionally providing a fastening element on the base to have the fastening element penetrate from an outside of the base into an inside of the base to abut the base positioner, so as to secure positioning of the base.

Upon placing the base 1a onto the tooth missing site in the patient oral area and becoming aware that the base 1a has a relatively large gap with respect to the neighboring teeth, the dentist may penetrate the above-discussed fastening element 14a from the outside of the base 1a into the interior of the base 1a to abut the base positioner 13a, and by doing so, a relative position of the base 1a with respect to the base positioner 13a can be fixed, making the base 1a not sliding relative to the base positioner 13a, and thus preventing oscillating and shaking of the base 1a in the gap. Once the computer-tomographic scanning is completed and it is determined that adjusting has to be done in a frontward or rearward direction, the above-discussed fastening element 14a can be released and adjusting of the base 1a can be done in either the frontward direction or the rearward direction to thereby have the base 1a adjusted and set to a better position.

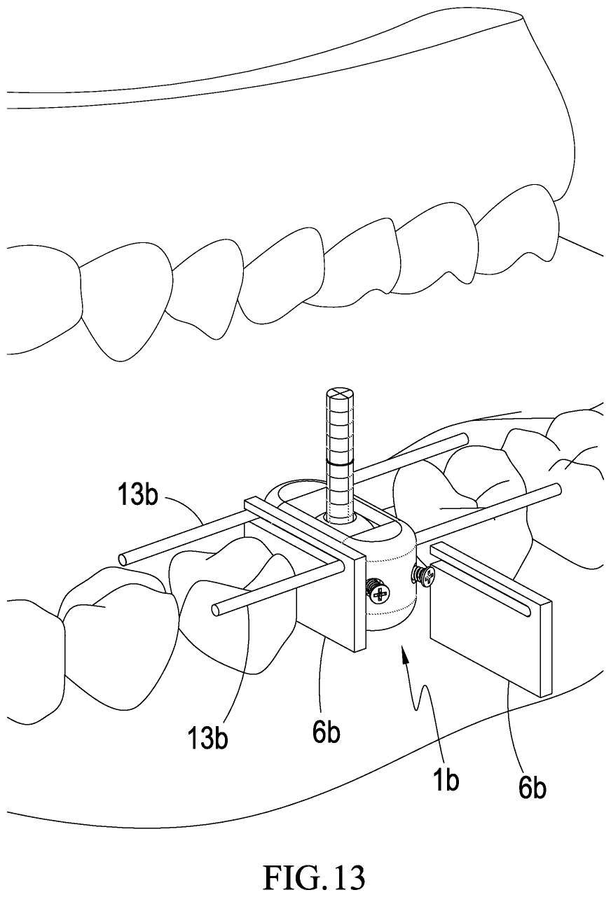

Referring to FIG. 13, the base positioner 13b is connectable with at least one filler board 6b, and the filler board 6b is positioned at one side of the base 1b. The filler board 6b is exemplified as being of a number of two, but is not limited thereto.

In the instant embodiment, Step (a1) can be carried out after Step (a) and is discussed as follows:

(a) placing a base on an implantation site in a patient oral portion, the base comprising a receiving space therein, the receiving space being provided therein with a sphere, the base being provided with at least one base positioner, and then, positioning the base positioner against neighboring teeth;

(a1) connecting the base positioner to at least one filler board arranged at one side of the base in order to secure positioning of the base.

The filler board 6b provides a function that is similar to that of the fastening element provided in the above embodiment to achieve an effect of positioning of the base 1b. When there is a relatively large gap existing between the base 1b and the neighboring teeth, the filler board 6b can be positioned and arranged in such a gap in order to have the gap between the base 1b and the neighboring teeth be filled up with the filler board 6b, making the base 1b not sliding relative to the base positioner 13b, and thus preventing oscillating and shaking of the base 1b in the gap. Once the computer-tomographic scanning is completed and it is determined that adjusting has to be done in a frontward or rearward direction, the above-discussed filler board 6b can be released or an additional one can be added to thereby achieve adjusting of the base 1a through sliding in either the frontward direction or the rearward direction to a better position. The instant embodiment is provided as an example of a different way of achieving securing of the base 1b for application in a different situation.

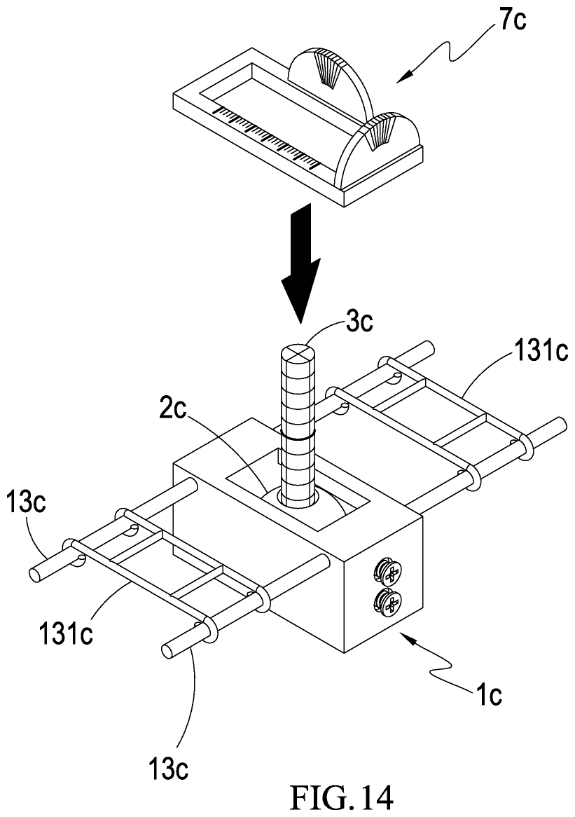

Referring to FIG. 14, the base positioner 13c comprises at least one positioning rib 131c, the positioning rib 131c being exemplified as being of a number of two, but not limited thereto. The base 1c is further provided with a measuring piece 7c, the measuring piece 7c being positioned to correspond to the above-discussed adjustor 3c, the measuring piece 7c being a kit that comprises a protractor and a scale. Further, in the instant embodiment, the base 1c is exemplified as being of a rectangular configuration, but not limited thereto.

In the instant embodiment, Step (e1) can be carried out after Step (e) and is discussed as follows:

(e) controlling an adjustor that is partly penetrating and arranged in the sphere and is not X-ray transmittable to drive the sphere for rotating and sliding in the receiving space in order to adjust the sphere to a correct position;

(e1) mounting a measuring piece on the base so that precise reading is acquired for the adjusting done with the adjustor to thereby move the sphere to achieve precise and accurate positioning.

In controlling the adjustor 3c to carry out adjusting of angle and position of the sphere 2c, the dentist may fit the measuring piece 7c to the base 1c, in order to allow the dentist to acquire more accurate and more precise reading for the adjusting, thereby achieving adjusting of the sphere 2c to the best position and the best angle, and as such, the above-discussed measuring piece 7c provides an effect of assisting of measurement.

Further, during placing the base 1c at the tooth missing site in the patient oral area and carrying out coating of the molding material, the dentist may use the structure of the above-discussed positioning ribs 131c to increase a contact area between the present invention and the molding material and that between the present invention and the tooth, so as to improve stability of mounting and dismounting of the tooth mold thereby improving operation accuracy.

Thus, the present invention provides an instantaneous dental implant surgery guiding device and a method thereof, which can improve the prior art based on the following key features:

Firstly, the adjustor 3 is controllable to drive the sphere 2 to rotate for adjusting the through hole 21 to correct position and angle, so as to enable a process of establishing an implant route, whereby time for mold removing, gypsum pouring, wax sculpturing, and repeated molding can be saved and the cost of retaining a technician workshop for fabrication of guide twice can also be saved, and thus, achieving effects of safe and accurate positioning, instantaneousness and convenience, time saving, and cost reduction.

Secondly, utilizing the structure of the fastening element 14a to secure the base 1a may avoid oscillating and shaking of the base 1a in a gap and also enable fine adjusting of the base 1a.

Thirdly, utilizing the structure of the filler board 6b to secure the base 1b may avoid oscillating and shaking of the base 1b in a gap and also enable fine adjusting of the base 1a.

Fourthly, utilizing the structure of the positioning rib 131c may increase the contact area between the present invention and a tooth to thereby improving stability and accuracy of operation.

Fifthly, utilizing the structure of the measuring piece 7c may improve accuracy and precision of adjusting, making it possible to achieve predetermined positioning safely with one-time operation during dental implanting.

Sixthly, based on the above, the latest dental digitization technique today is that an oral scanning machine and computer tomography are used in a clinic for transmission of digital files to a digitalized dental technician, and a surgical guide can be made through three-dimensional printing for being then sent back to the clinic. Doing mailing back and forth is quite time consuming, and nowadays, the oral scanning machine is expensive, and also requires a skilled operator to operate the machine and software. To establish skilled expertise and all sorts of digitalized equipment in a clinic would require labor, facility, and expenditure that are generally not affordable by an ordinary dental clinic. Thus, through the present invention and the method thereof, it is possible to realize, in a manner of time saving, safe and convenient way, a function of fabrication of surgical guides similar to those made through digitalized dentistry.

I claim:

1. An instantaneous dental implant surgery guiding device, mainly comprising:
a base, the base comprising a receiving space therein;
a sphere, the sphere being arranged in the receiving space, the sphere comprising a through hole formed therein;
an adjustor, the adjustor partly penetrating and arranged in the through hole;
a plurality of sphere positioners, each of the sphere positioners being arranged at one of two sides of the base and penetrating from outside the base into the receiving space to abut against the sphere; and
at least one base positioner, the base positioner being arranged on the base.

2. The instantaneous dental implant surgery guiding device according to claim 1, wherein the base positioner comprises at least one positioning rib provided thereon.

3. The instantaneous dental implant surgery guiding device according to claim 1, wherein the base positioner is connected to at least one filler board, the filler board being arranged at one side of the base.

4. The instantaneous dental implant surgery guiding device according to claim 1, wherein the base is provided with a fastening element, the fastening element being arranged at one side of the base, the fastening element penetrating from outside of the base into interior of the base and abutting the base positioner.

5. The instantaneous dental implant surgery guiding device according to claim 1, wherein the base is provided with a measuring piece, the measuring piece corresponding, in position, to the adjustor.

6. A method of operating an instantaneous dental implant surgery guiding device, comprising the following steps:
(a) placing a base on an implantation site in a patient oral portion, the base comprising a receiving space therein, the receiving space being provided therein with a sphere, the base being provided with at least one base positioner, and then, positioning the base positioner against neighboring teeth;
(b) applying a glue to initially retain the base positioner, then coating a molding material on and covering the base positioner and the neighboring teeth, and waiting until curing of the molding material;
(c) carrying out computer-tomographic scanning on the patient oral area, and determining whether or not to carry out adjusting of the sphere in respect of angle and position;
(d) moving a plurality of sphere positioners that are arranged on the base and abutting the sphere to have each of the sphere positioners moved away from the sphere, in order to carry out adjusting of the sphere;
(e) controlling an adjustor that is partly penetrating and arranged in the sphere and is not X-ray transmittable to drive the sphere for rotating and sliding in the receiving space in order to adjust the sphere to a correct position;
(f) moving each of the sphere positioners to re-abut the sphere that is at the correct position in order to achieve an effect of positioning of the sphere; and
(g) removing the adjustor out of a through hole formed in the sphere, and applying an implant driller to drill, along the angle-and-position-adjusted through hole, into an implantation site in the patient oral portion in order to obtain a correct expanded and implanting router, and finally, smoothly and safely placing an implant to the predetermined position.

7. The method of operating the instantaneous dental implant surgery guiding device according to claim 6, wherein the base positioner comprises at least one positioning rib provided thereon.

8. The method of operating the instantaneous dental implant surgery guiding device according to claim 6, wherein Step (a1) is carried out after Step (a) by connecting the base positioner to at least one filler board arranged at one side of the base in order to secure positioning of the base.

9. The method of operating the instantaneous dental implant surgery guiding device according to claim 6, wherein Step (a1) is carried out after Step (a) by additionally providing a fastening element on the base to have the fastening element penetrate from an outside of the base into an inside of the base to abut the base positioner, so as to secure positioning of the base.

10. The method of operating the instantaneous dental implant surgery guiding device according to claim 6, wherein Step (e1) is carried out after Step (e) by mounting a measuring piece on the base so that precise reading is acquired for the adjusting done with the adjustor to thereby move the sphere to achieve precise and accurate positioning.
